(19) United States
(12) Patent Application Publication
(10) Pub. No.: US 2004/0071751 A1
(43) Pub. Date: Apr. 15, 2004

(54) Blood neutral fat concentration controlling agent and method of controlling neutral fat concentration in blood

(75) Inventors: Hideaki Maki, Yokosuka-shi (JP); Michio Kasai, Yokosuka-shi (JP); Naohisa Nosaka, Yokosuka-shi (JP); Mitsuko Okazaki, Sakado-shi (JP); Osamu Igarashi, Hitachi-shi (JP); Kazuo Kondo, Tokyo (JP)

Correspondence Address: BURNS, DOANE, SWECKER & MATHIS, L.L.P., P.O. Box 1404, Alexandria, VA, 22313-1404, US

(73) Assignee: THE NISSHIN OILLIO, LTD.

(21) Appl. No.: 10/464,865

(22) Filed: Jun. 19, 2003

(30) Foreign Application Priority Data
Jul. 10, 2002 (JP) 2002-200844

(51) Int. Cl. A61K 31/56; A61K 47/00; A61K 31/355; A61K 31/225

(52) U.S. Cl. 424/439; 514/458; 514/171; 514/547

(57) ABSTRACT

The present invention provides a blood neutral fat concentration controlling agent containing medium-chain triglycerides, wherein at least 90% by mass of the fatty acids constituting the medium-chain triglycerides are saturated fatty acids having 8 and 10 carbon atoms, a mass ratio of the saturated fatty acid having 8 carbon atoms to the saturated fatty acid having 10 carbon atoms is 60/40 to 85/15, and an amount of the saturated fatty acid having 8 carbon atoms is 60 to 85% by mass based on the total fatty acids bonded to the 2-position of the triglycerides, and also foods for controlling blood neutral fat concentration, which contain the agent. According to the present invention, the neutral fat concentration in human blood can be easily controlled without dieting and without fear of side effects.

### BACKGROUND OF THE INVENTION

The present invention relates to a composition for controlling neutral fat concentration in blood. In particular, the present invention relates to a blood neutral fat concentration controlling agent capable of safely controlling an increased neutral fat concentration in human blood in a proper range and foods for controlling neutral fat concentration in blood, which contains the blood neutral fat concentration controlling agent. The present invention also relates to a method of controlling neutral fat concentration in blood.

Fats in blood include neutral fat (triglycerides), cholesterol, phospholipids and free fatty acids. When the fat concentration in blood is abnormally high, such a case is called “hyperlipemia”. Hyperlipemia is one of “quartet for death” which must be carefully treated like lowering of glucose tolerance, adiposity and high blood pressure. It is also well known that by the synergistic effect of these risk factors, various diseases including circulatory diseases such as arteriosclerosis and ischemic heart disease are caused. In particular, blood neutral fat (triglycerides) and blood cholesterol were suggested to be risk factors for causing ischemic heart disease and coronary artery disease. In the clinical field, among hyperlipemia patients, hypertriglyceridemia patients having a high neutral fat (triglyceride) level and also hypercholesterolemia patients having a high cholesterol level are serious problems. For example, according to the results of the national nutrition survey of the Ministry of Health, Labour and Welfare, 32.8% of the adults are suffering from hypertriglyceridemia and 30.6% of them are suffering from hypercholesterolemia (on the basis of the diagnosis standard according to the hyperlipemia treatment guideline of Japan Atherosclerosis Society). Thus, hypercholesterolemia is a problem close to not only European and American people but also Japanese people (refer to page 113 of a report of “The Present Nutritional Situation of Japan—The Results of the National Nutrition Survey—edited by Kenko Eiyo Joho Kenkyu-kai and published by Daiichi Shuppan in 2001).

Under these circumstances, according to the hypercholesterolemia treatment guideline of Japan Atherosclerosis Society, the diagnosis standard level and the therapeutic threshold for hyperlipemia based on the blood fat is a neutral fat (triglycerides) concentration of 150 mg/dL or total cholesterol concentration of 220 mg/dL “Doumyakukouka (Atherosclerosis)” Vol. 25, pages 1 to 34, 1997). Hyperlipemia is generally caused by obesity or overweight due to overeating or lack of exercise excepting cases of heredity or illness. Therefore, the treatment of hyperlipemia starts with diet therapy. The diet therapy in which meals of a limited calorie are given is not easy for the patients because it involves a mental difficulty. In addition, an excess diet therapy might cause troubles such as malnutrition. Further, there is a danger of causing a pathologic symptom called “anorexia”. Another treatment method is to reduce the blood fat concentration by the administration of a medicine. However, side effects of the medicine must be taken into consideration in addition to the effects thereof in this method. Thus, this method cannot be considered to be a simple and easy method for reducing the blood fat concentration for each patient.

To find a way out of the present difficulty, low-calorie oils and fats (substitutes for fat) have been developed. However, none of them is satisfactory in their safety, physical properties, suitability for cooking, flavor and taste at present. For example, it was disclosed that sucrose fatty acid esters are usable as low-calorie oil because they are not absorbed in the digestive tract but excreted in feces (the specification of U.S. Pat. No. 3,600,186). In U.S.A., the use of the sucrose fatty acid esters for salty snacks was permitted, and potato chips containing the sucrose fatty acid esters were already put on the market. However, it is a duty to indicate that commercial products containing the sucrose fatty acid esters might cause abdominal convulsion and a loose passage and that they inhibit the absorption of fat-soluble vitamins. The energy density of proteins and carbohydrates is less than a half of that of fats. In this connection, it is known that by processing protein and carbohydrates to attach physical properties, flavor and taste of fats to them, fat substitutes having a low calorie can be obtained “Eiyougaku Review (Nutrition Review)”, Vol. 4, No. 4, pages 23 to 33, 1996). By using such fat substitutes, ice creams and desserts having a low calorie can be prepared. However, these fat substitutes have defects that their flavor and taste are not good and that they are not resistant to heat and, therefore, they are unsuitable for the cooking by heat. Further, the fat substitutes themselves do not have the function of suitably controlling the fat concentration in blood.

Medium-chain triglycerides have been used as solvents for oil-soluble flavor for foods and colorants, releasing oils and lubricating oils for foods or starting materials for medicines, taking advantage of their high oxidation stability, low solidifying point, low viscosity, high colorlessness and high transparency and high solubility. The medium-chain triglycerides are also used as starting materials for enteric nutrients for efficiently supplying the energy, taking advantage of their high absorbability and energy ratio higher than that of saccharides.

It was reported that by replacing oils and fats in the meals with the medium-chain triglycerides, an effect of controlling the blood neutral fat concentration (effect of controlling the increase thereof) after meals is obtained (Calabrese, C. et al., Alternative Medicine Review, Vol. 4, pages 23 to 28, 1999). However, it has not yet been elucidated that the medium-chain triglycerides have a function of inhibiting the increase in the blood neutral fat concentration which depends on the physique of each person. Further, the expression of a function of controlling the blood neutral fat concentration by the bonding position of the fatty acid residue has not yet been elucidated at all.

### SUMMARY OF THE INVENTION

The object of the present invention is to provide an agent for controlling the blood neutral fat concentration which is easily usable without dieting and without a fear for the side effects.

Another object of the present invention is to provide a food for controlling the blood neutral fat concentration which contains the above-described agent for controlling the blood neutral fat concentration.

Still another object of the present invention is to provide a method of controlling the neutral fat concentration in the human blood.

The present invention has been attained on the basis of a finding that medium-chain triglycerides have a function of controlling the blood neutral fat concentration.

Namely, the present invention provides a blood neutral fat concentration controlling agent containing medium-chain triglycerides, wherein at least 90% by mass of the fatty acids constituting the medium-chain triglycerides are saturated fatty acids having 8 and 10 carbon atoms, a mass ratio of the saturated fatty acid having 8 carbon atoms to the saturated fatty acid having 10 carbon atoms is 60/40 to 85/15, and an amount of the saturated fatty acid having 8 carbon atoms is 60 to 85% by mass based on the total fatty acids bonded to the 2-position of the triglycerides.

The present invention provides a food for controlling the blood neutral fat concentration which contains the above-described agent for controlling the blood neutral fat concentration.

The present invention also provides a method of controlling neutral fat concentration in blood, which comprises administering the blood neutral fat concentration controlling agent to human beings.

### BRIEF DESCRIPTION OF THE DRAWINGS

FIG. 1 is a graph showing the area under the neutral fat concentration in blood/hour curve (a group having BMI of 23 or above) determined until 6 hours after taking food 1 for controlling the neutral fat concentration in blood.

FIG. 2 is a graph showing the area under the neutral fat concentration in blood/hour curve (a group having BMI value of below 23) determined until 6 hours after taking food 1 for controlling the neutral fat concentration in blood.

### BEST MODE FOR CARRYING OUT THE INVENTION

The term “medium-chain triglycerides” herein indicates triglycerides wherein the constitutional fatty acids are medium-chain fatty acids, namely triacylglycerols. The medium-chain fatty acids herein are preferably fatty acids having 6 to 12 carbon atoms, particularly saturated fatty acids, and more preferably saturated fatty acids having an even number of carbon atoms. Examples of those saturated fatty acids include caproic acid, caprylic acid, capric acid and lauric acid. Among them, caprylic acid and capric acid which are saturated fatty acids having 8 to 10 carbon atoms are preferred. More preferably, at least 90% by mass (desirably 95 to 100%) of fatty acids constituting the medium-chain triglycerides are saturated fatty acids having 8 and 10 carbon atoms, the mass ratio of the saturated fatty acid having 8 carbon atoms to the saturated fatty acid having 10 carbon atoms is 60/40 to 85/15 (desirably 7/3 to 8/2), and the amount of the saturated fatty acid having 8 carbon atoms is 60 to 85% (desirably 65 to 80%) by mass based on the total fatty acids bonded at the 2-position of the triglycerides.

Processes for producing such medium-chain triglycerides are not particularly limited. For example, the medium-chain triglycerides can be produced by the esterification of medium-chain fatty acids from palm kernel oil or coconut oil and glycerol. Although the esterification conditions are also not particularly limited, such a medium-chain triglyceride can be obtained by, for example, the reaction carried out in the absence of catalyst without any solvent under pressure. Of course, such medium-chain triglycerides can be obtained by the reaction carried out in the presence of a catalyst in a solvent. The medium-chain triglycerides, wherein at least 90% by mass of the constituting fatty acids are saturated fatty acids having 8 and 10 carbon atoms, the mass ratio of the saturated fatty acid having 8 carbon atoms to the saturated fatty acid having 10 carbon atoms is 60/40 to 85/15, and the amount of the saturated fatty acid having 8 carbon atoms is 60 to 85% by mass based on the fatty acids bonded at the 2-position of the triglycerides can be obtained by mixing the medium-chain triglycerides with the medium-chain fatty acids in a predetermined mixing ratio in the presence of a lipase having a position specificity such as “Lipase PL” (a product of Meito Sangyo Co., Ltd.). The medium-chain triglycerides per se can be obtained from oilseeds of a recombinant vegetable. Further, the medium-chain triglycerides can be produced from medium-chain fatty acids obtained from the oilseeds of a recombinant vegetable.

The blood fats in the present invention involve neutral fat, cholesterol, phospholipids and free fatty acids. The neutral fat in the blood is triglycerides contained in blood, particularly triglycerides in the serum. The serum is a supernatant liquid obtained by leaving blood to stand in a test tube, the blood being taken for analyzing the lipids in blood. The serum is a component of blood, which remains after removing blood cells and platelets from blood.

The term “control of neutral fat concentration in blood” herein indicates the function of controlling the neutral fat concentration in blood by controlling the postprandial increase of the neutral fat concentration in blood when it increases by the intake of a lipid such as a widely used oil (for example, mixed salad oil) at a meal or by exerting no influence on the neutral fat concentration in blood when it is normal. The term “postprandial or after meals” herein indicates “after the intake of a lipid such as a vegetable oil or fat at a meal”.

The agent for controlling the neutral fat concentration in blood of the present invention contains desirably at least 5% by mass, more desirably 5 to 100% by mass, most preferably at least 40% by mass, in particular, 40 to 99.95% by mass, of the medium-chain triglycerides.

The present invention also provides a method of controlling neutral fat concentration in human blood, which comprises administering the above-described agent for controlling blood neutral fat concentration to the human beings. The method of the administration to the human beings is to administer the agent for controlling blood neutral fat concentration per se to them or to feed foods to which such an agent was added. In this method, 1 to 25 g of the medium-chain triglycerides are given each time or each meal to the human beings.

By the method of the present invention of controlling the neutral fat concentration in blood, an increase in the neutral fat concentration in blood after meals, by the intake of fats from the foods, can be controlled and the neutral fat concentration in blood can be kept in a suitable range. The method of the present invention of controlling the neutral fat concentration in blood is particularly suitable for patients having a BMI of at least 23.

The blood neutral fat concentration controlling agent of the present invention can contain components other than the medium-chain triglycerides. Those components are not particularly limited and they include ordinary cooking oils such as soybean oil, rapeseed oil, corn oil, sesame oil, sesame salad oil, perilla oil (Shiso-yu), linseed oil, peanut oil, safflower oil, safflower oil having a high oleic acid content, sunflower oil, sunflower oil having a high oleic acid content, cotton seed oil, grape seed oil, macadamia nut oil, hazelnut oil, pumpkin nut oil, walnut oil, tsubaki oil, tea nut oil, perilla oil (Egoma-yu), borage oil, olive oil, rice bran oil, wheat germ oil, palm oil, palm kernel oil, coconut oil, cacao butter, beef tallow, lard, chicken fat, milk fat, fish oil, seal oil and algae oil; as well as those oils and fats having a saturation degree lowered by the improvement of species of them, the hydrogenated oils and fats and fractionated oils and fats. Further, they can contain vitamin E and phytosterols as well as polyglycerol fatty acid esters, sucrose fatty acid esters, sorbitan fatty acid esters, ascorbic acid fatty acid esters, lignan, coenzyme Q, phospholipids, oryzanols and diglycerides.

In those components, one or more components selected from the group consisting of animal and vegetable oils, vitamin E and phytosterols are preferred. When the animal and vegetable oils are contained in the agent, the amount of them is preferably not larger than 60% by mass, more preferably 5 to 55% by mass and particularly preferably 5 to 50% by mass based on the whole composition. When vitamin E is contained therein, the amount thereof is preferably 0.01 to 5% by mass, more preferably 0.01 to 2% by mass, based on the whole composition. When phytosterols are contained, the amount thereof is preferably 0.5 to 6% by mass, more preferably 1 to 4% by mass, based on the whole composition.

BMI (Body Mass Index) herein is a numeral used for the medical judge of obesity. The details of BMI are described on pages 14 to 24 of “Himansho: Shindan•Chiryo•Shido no Tebiki (Obesity: Handbook of Diagnosis, Treatment and Guidance)” edited by The Editorial Committee on the Handbook of Diagnosis of Obesity of The Japan Society for the Study of Obesity and published by Ishiyaku Shuppan on 1993). BMI (kg/m²) is calculated by the formula: (body weight, kg)/(height, m)/(height, m). The Japan Society for the Study of Obesity proposed that the standard index of BMI is 22 according to the results of epidemiology research of Japanese people. Because BMI can be easily calculated from the measured body weight and height, it is generally employed mainly for measuring the body fat instead of a body density method (Hydrodensitometry or air displacement method), internal K⁴⁰ determination method or impedance method. The lower BMI, the thinner and the lower the body fat content, i.e. the lower the degree of obesity. On the contrary, the higher BMI, the higher the body fat content or, in other words, the higher the degree of obesity. It has been made apparent by studies that as the degree of obesity is elevated or, in other words, as BMI is increased, the neutral fat concentration in blood and cholesterol in blood are increased [refer to: Seiichiro Tarui et al., Seijin Himan, Shoni Himan no Seiin to Byotai ni kansuru Sougouteki Kenkyu (Comprehensive Study of the Cause and Pathology of Adult Obesity and Child Obesity) in Kaken Sougou Kenkyu Seika Houkokusno in 1984 (The Report on the Total Study Results in 1984 by the Ministry of Health and Welfare) published in 1985].

When the neutral fat concentration in blood is elevated by the intake of lipids through a meal, the increase in the neutral fat concentration in blood can be controlled by using the blood neutral fat concentration controlling agent of the present invention within 6 hours after the meal particularly when BMI is 23 or above. When the dynamics of the neutral fat concentration in blood are normal after the meal, and particularly when BMI is below 23, the agent of the present invention exerts no influence on the neutral fat concentration in blood.

In the present invention, the form of the agent for controlling the neutral fat concentration in blood is not particularly limited. It may be in the form of capsules, tablets or the like, or it may be added to foods, processed foods, drinks, seasonings, cakes, etc. For example, the agent for controlling the neutral fat concentration in blood can be added to gelatin capsules, cooking oils, dressings, margarines, processed margarines, fat spreads, creams, ice creams, mayonnaises, breads, cakes, doughnuts, muffins, scones, deep-fried foods, snacks, liquid diets, etc. The agent of the present invention is added to those foods in such an amount that the medium-chain triglyceride content of the foods will be preferably at least 5% by mass, more preferably 6 to 100% by mass.

The following Examples will further specifically illustrate the present invention, which by no means limit the invention.

### EXAMPLE 1

500 g of rapeseed oil produced by The Nisshin Oil Mills, Ltd. was mixed in 500 g of medium-chain triglycerides (trade name: ODO) produced by The Nisshin Oil Mills, Ltd. to obtain blood neutral fat concentration controlling agent 1. Separately, 100 g of lard produced by Snow Brand Milk Products Co., Ltd. was mixed in 900 g of medium-chain triglycerides (trade name: ODO) produced by The Nisshin Oil Mills, Ltd. to obtain blood neutral fat concentration controlling agent 2. 99.9% by mass of fatty acids constituting the medium-chain triglycerides comprised saturated fatty acids having 8 and 10 carbon atoms, and the ratio by mass of the saturated fatty acid having 8 carbon atoms to the saturated fatty acid having 10 carbon atoms was 7.4:2.6. In the fatty acid composition bonded to the 2-position of the triglyceride, the relative amount of the saturated fatty acid having 8 carbon atoms was 73% by mass. The relative amount of the fatty acids bonded to the 2-position of the triglyceride was determined by Brockerhoff method (Brockerhoff, H., Journal of Lipid Research, Vol. 6, pages 10 to 15, 1965).

### EXAMPLE 2

1 g of tocopherol (trade name: Tocopherol 85) produced by The Nisshin Oil Mills, Ltd. was added to 999 g of medium-chain triglycerides (trade name: ODO) produced by The Nisshin Oil Mills, Ltd., and they were thoroughly stirred to obtain a solution, i. e. blood neutral fat concentration controlling agent 3. 99.9% by mass of fatty acids constituting the medium-chain triglycerides comprised saturated fatty acids having 8 and 10 carbon atoms, and the ratio by mass of the saturated fatty acid having 8 carbon atoms to the saturated fatty acid having 10 carbon atoms was 7.4:2.6. In the fatty acid composition bonded to the 2-position of the triglyceride, the relative amount of the saturated fatty acid having 8 carbon atoms was 73% by mass.

### EXAMPLE 3

20 g of vegetable sterol derived from rice oil was added to 980 g of blood neutral fat concentration controlling agent 1, and they were thoroughly mixed by stirring to obtain blood neutral fat concentration controlling agent 4.

### EXAMPLE 4

An oil-and-fat drink (blood neutral fat concentration controlling food 1) was prepared from blood neutral fat concentration controlling agent 3 produced in Example 2 as follows: The blood neutral fat concentration controlling food 1 contained 10 g of the blood neutral fat concentration controlling agent 3.

Composition (Parts by Mass)

| | |
|---|---|
| Dextrin | 25 parts |
| Casein | 13 parts |
| Refined white sugar | 2 parts |
| Blood neutral fat concentration controlling agent 3 | 10 parts |
| Water | 150 parts |

Production Method

Preparation: Dextrin, casein, refined white sugar and blood neutral fat controlling agent 3 were added to warm water kept at 60° C. and they were mixed with a homomixer. The obtained mixture was then emulsified with a homogenizer. The obtained emulsion was sealed in a vessel and then sterilized at 121° C. for 20 minutes.

### EXAMPLE 5

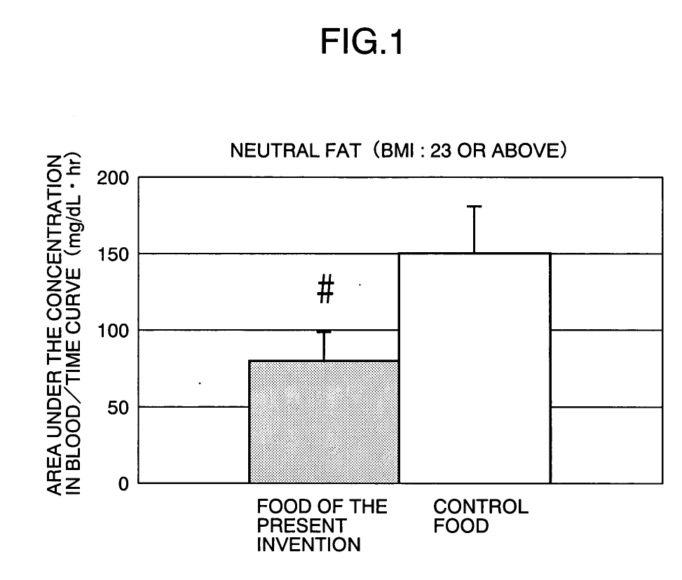

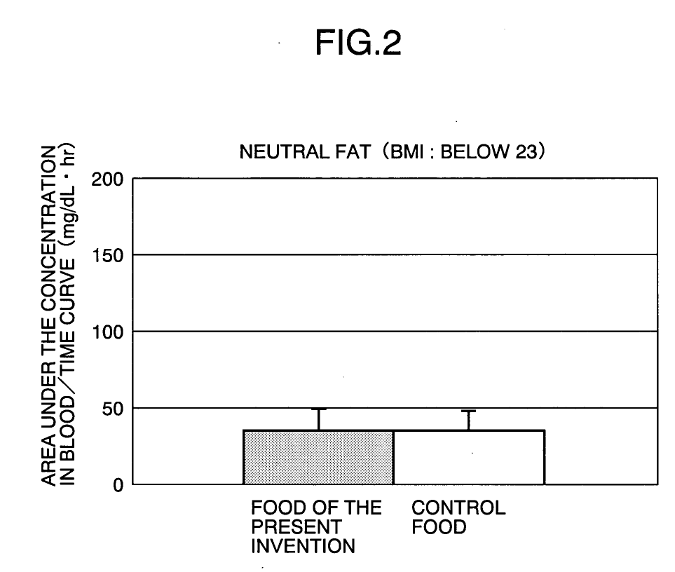

Tests on the effect of controlling the blood neutral fat concentration were conducted by crossover method by using blood neutral fat concentration controlling food 1 obtained in Example 4. The foods of 25 subjects were controlled for 3 days before the tests. After the fasting for 12 hours started on the day before the start of the tests, blood neutral fat concentration controlling food 1 was given to the subjects in the morning on the first day of the tests. An oil-and-fat drink containing the equal amount of a mixed salad oil (rapeseed oil/soybean oil in mass ratio of 7/3) was used as a control. 2, 4 and 6 hours after taking the oil-and-fat drink, blood samples were taken to determine the blood neutral fat concentration. The effects were evaluated from the area under the concentration in blood/time curve calculated from the blood neutral fat concentration by the trapezoidal method. The test results are shown in Table 1 and FIGS. 1 and 2. The results were statistically dealt by dividing BMI values into a group of 23 or higher and a group of below 23. Generally, the area under the concentration in blood/time curve is a function of the internal dynamics and it indicates the amount of the administered substance which was absorbed in the body per a unit time. This is calculated by the trapezoidal method or exponential method.

TABLE 1
Test results of blood neutral fat concentration controlling food 1
(actual measurements; the real number is average ± standard error)

| | Time after | | | | |
|---|---|---|---|---|---|
| | taking oil- | Control food | | Food of the present invention | |
| | and-fat drink | BMI: 23 or | BMI: | BMI: 23 or | BMI: below |
| | (hours) | above | below 23 | above | 23 |
| Neutral | 0 | 177.3 ± 30.9* | 81.9 ± 10.8 | 154.2 ± 26.4* | 78.5 ± 7.4 |
| fat | 2 | 212.9 ± 31.7 | 91.4 ± 11.8 | 160.2 ± 25.4*# | 77.8 ± 7.3 |
| (mg/dL) | 4 | 213.0 ± 36.1 | 88.3 ± 12.7 | 175.6 ± 27.1*# | 85.5 ± 8.4 |
| | 6 | 179.4 ± 35.1 | 75.7 ± 11.4 | 174.1 ± 25.4* | 84.2 ± 8.3 |

*A statistically significant difference from a group having BMI of below 23 was recognized in the groups that took the same foods (level of significance: 5% or below).
#A statistically significant difference from a group which took the control food was recognized in the groups of the same BMI (level of significance: 5% or below)

As shown in Table 1 and FIGS. 1 and 2, it is apparent that the postprandial fat concentration in blood of the subjects having a BMI of 23 or higher was significantly lowered by taking blood neutral fat concentration controlling food 1 of the present invention. Even when the subjects having a BMI of below 23 took blood neutral fat concentration controlling food 1 of the present invention, the change in the blood neutral fat concentration was the same as that observed after taking the control food. Namely, no influence was exerted in this case. From those facts, it was apparent that blood neutral fat concentration controlling food 1 containing the blood neutral fat concentration controlling agent of the present invention controls the postprandial increase in neutral fat concentration in blood of patients having BMI of 23 or above. It was also apparent that this food 1 exerts no influence on the neutral fat concentration in blood of subjects having a proper neutral fat concentration in blood.

The present invention can provide the highly effective, safe and easily usable agent for controlling neutral fat concentration in blood and also foods containing the agent for controlling neutral fat concentration in blood. In particular, by using the agent for controlling blood neutral fat concentration of the present invention, the postprandial increase of the neutral fat concentration in blood of patients having a BMI of 23 or above can be controlled. Therefore, an effect of preventing habitual diseases such as hyperlipemia of overweight people called “obesity reserves” can be expected. On the other hand, the blood neutral fat concentration controlling agent of the present invention exerts no influence on the blood neutral fat concentration of human beings having normal dynamics of the postprandial neutral fat concentration in blood or, in particular, having a BMI of below 23.

What is claimed is:

1. A blood neutral fat concentration controlling agent containing medium-chain triglycerides, wherein at least 90% by mass of the fatty acids constituting the medium-chain triglycerides are saturated fatty acids having 8 and 10 carbon atoms, a mass ratio of the saturated fatty acid having 8 carbon atoms to the saturated fatty acid having 10 carbon atoms is 60/40 to 85/15, and an amount of the saturated fatty acid having 8 carbon atoms is 60 to 85% by mass based on the total fatty acids bonded to the 2-position of the triglycerides.

2. The blood neutral fat concentration controlling agent according to claim 1, which has a medium-chain triglyceride content of at least 5% by mass.

3. The blood neutral fat concentration controlling agent according to claim 1, which contains at least 40% by mass of the medium-chain triglycerides.

4. The blood neutral fat concentration controlling agent according to claim 1, which contains an animal or vegetable oil.

5. The blood neutral fat concentration controlling agent according to claim 1, which further contains vitamin E.

6. The blood neutral fat concentration controlling agent according to claim 1, which further contains phytosterols.

7. The blood neutral fat concentration controlling agent according to claim 1, which is used for controlling blood neutral fat concentration of patients having a BMI of 23 or higher.

8. Foods for controlling blood neutral fat concentration, which contain the blood neutral fat concentration controlling agent according to claim 1.

9. Foods for controlling blood neutral fat concentration according to claim 8, which are cooking oils, mayonnaises, margarines, processed margarines, fat spreads, dressings, breads, ice creams, creams, cakes, doughnuts, muffins, scones or deep-fried foods.

10. A method of controlling neutral fat concentration in blood of human beings, which comprises administering a blood neutral fat concentration controlling agent containing medium-chain medium triglycerides, wherein at least 90% by mass of the fatty acids constituting the medium-chain triglycerides are saturated fatty acids having 8 and 10 carbon atoms, a mass ratio of the saturated fatty acid having 8 carbon atoms to the saturated fatty acid having 10 carbon atoms is 60/40 to 85/15, and an amount of the saturated fatty acid having 8 carbon atoms is 60 to 85% by mass based on the total fatty acids bonded at the 2-position of the triglyceride, to the human beings.

11. The method according to claim 10, wherein the medium-cnain triglyceride content of the blood neutral fat concentration controlling agent is at least 5% by mass.

12. The method according to claim 10, wherein the medium-chain triglyceride content of the blood neutral fat concentration controlling agent is at least 40% by mass.

13. The method according to claim 10, wherein the blood neutral fat concentration controlling agent contains an animal or vegetable oil.

14. The method according to claim 10, wherein the blood neutral fat concentration controlling agent further contains vitamin E.

15. The method according to claim 10, wherein the blood neutral fat concentration controlling agent further contains phytosterols.

16. The method according to claim 10, wherein the patients are those having a BMI of 23 or higher.

17. The method according to claim 10, wherein foods containing the blood neutral fat concentration controlling agent are administered to the human beings.

18. The method according to claim 10, wherein the foods are selected from the group consisting of cooking oils, mayonnaises, margarines, processed margarines, fat spreads, dressings, breads, ice creams, creams, cakes, doughnuts, muffins, scones and deep-fried foods.
